(19) United States
(12) Patent Application Publication
(10) Pub. No.: US 2008/0188421 A1
(43) Pub. Date: Aug. 7, 2008

(54) Hunter-Killer Peptides and Methods of Use

(76) Inventors: Dale E. Bredesen, Novato, CA (US); Michael Ellerby, Novato, CA (US); Lisa Ellerby, Novato, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/795,703

(22) PCT Filed: Mar. 31, 2005
(86) PCT No.: PCT/US2005/010951

§ 371 Date: Jul. 19, 2007

Related U.S. Application Data
(60) Provisional application No. 60/558,448, filed on Mar. 31, 2004.

(51) Int. Cl. A61K 38/10; C07K 7/00; A61K 38/08; A61P 35/00

(52) U.S. Cl. 514/14; 530/330; 530/329; 530/327; 514/17; 514/16

(57) ABSTRACT

The present invention provides homing conjugates containing an antimicrobial peptide and a tumor homing molecule, wherein the tumor homing molecule comprises a dimer of two endothelium-homing peptide monomers, wherein the conjugate homes to and is internalized by a tumor cell type or tissue comprising angiogenic endothelial cells and exhibits high toxicity thereto, wherein the high toxicity is due to disruption of mitochondrial membranes, and wherein the antimicrobial peptide has low mammalian cell toxicity when not linked to said tumor homing molecule. The present invention is based, in part, on the discovery that dimerization of endothelium-homing peptide monomer confers greatly increased cytotoxic activity on the conjugate. Based on this discovery, the invention further provides methods of inducing selective toxicity in vivo in an angiogenic endothelial tissue or cell type as well as methods of treating an individual having cancer by administering an effective amount of a homing conjugate of the invention also are provided.

Thus, under normal physiological conditions, the growth of new microvessels is carefully regulated.

Pathologic angiogenesis also is a focal process, yet persists for months or years. Tumors, for example, are characterized by a relatively high level of active angiogenesis, resulting in the continual formation of new blood vessels to support the growing tumor. The ability of a tumor to induce the proliferation of new blood vessels has a profound effect on its growth and metastasis, with rapid expansion of a tumor cell population following the onset of angiogenic activity. In contrast, the absence of angiogenic activity limits tumors to a few million cells in a volume of a few cubic millimeters; primary tumors or metastases that are not angiogenic generally are not clinically detectable. Thus, antiangiogenic therapy would be extremely useful, for example, in limiting tumor size and metastasis. Antiangiogenic therapy similarly would be useful in treating other disorders involving pathologic angiogenesis, such as diseases of ocular neovascularization, arthritis, atherosclerosis and endometriosis.

A major hurdle to advances in treating is the relative lack of agents that can selectively target the cancer, while sparing normal tissue. For example, radiation therapy and surgery, which generally are localized treatments, can cause substantial damage to normal tissue in the treatment field, resulting in scarring and, in severe cases, loss of function of the normal tissue. Chemotherapy, which generally is administered systemically, can cause substantial damage to organs such as bone marrow, mucosae, skin and the small intestine, which undergo rapid cell turnover and continuous cell division. As a result, undesirable side effects, for example, nausea, hair loss and reduced blood cell counts, occur as a result of systemically treating a cancer patient with chemotherapeutic agents. Such undesirable side effects often limit the amount of a treatment that can be administered. Due to such shortcomings in treatment, cancer remains a leading cause of patient morbidity and death.

Potent antimicrobial activity has been observed for a class of peptides including naturally occurring peptides such as melittin, the gramicidins, magainins, defensins and cecropins. Naturally occurring antimicrobial peptides, and related synthetic antimicrobial sequences, generally have an equivalent number of polar and nonpolar residues within an amphipathic domain and a sufficient number of basic residues to give the peptide an overall positive charge at neutral pH. The biological activity of amphipathic α-helical peptides against Gram-positive bacteria may result from the ability of these peptides to form ion channels through membrane bilayers. Many antimicrobial peptides selectively inhibit and kill bacteria while maintaining low mammalian cell cytotoxicity, with the differential sensitivity of bacterial cells apparently due to membrane differences between bacteria and mammalian cells. As shown herein, these antimicrobial peptides can be endowed with selective cytotoxic activity against a particular eukaryotic cell type, such as angiogenic endothelial cells.

Thus, there is a need for novel anti-cancer therapeutics that can selectively target the angiogenic endothelial cells. The present invention satisfies this need and provides related advantages.

### SUMMARY OF THE INVENTION

The present invention provides homing conjugates containing an antimicrobial peptide and a tumor homing molecule, wherein the tumor homing molecule comprises a dimer of two endothelium-homing peptide monomers, wherein the conjugate homes to and is internalized by a tumor cell type or tissue comprising angiogenic endothelial cells and exhibits high toxicity thereto, wherein the high toxicity is due to disruption of mitochondrial membranes, and wherein the antimicrobial peptide has low mammalian cell toxicity when not linked to said tumor homing molecule. The present invention is based, in part, on the discovery that dimerization of endothelium-homing peptide monomer confers greatly increased cytotoxic activity on the conjugate. Based on this discovery, the invention further provides methods of inducing selective toxicity in vivo in an angiogenic endothelial tissue or cell type as well as methods of treating an individual having cancer by administering an effective amount of a homing conjugate of the invention also are provided.

### BRIEF DESCRIPTION OF THE DRAWINGS

FIG. 1 shows a schematic representation of the HK-1 homing conjugate of the invention having the sequence (CNGRC-GG-_{d}(KLAKLAK)₂)₂ (SEQ ID NO: 9). This homing conjugate consists of two endothelium-homing peptide monomers having the sequence CNGRC (SEQ ID NO: 1), each monomer linked to the antimicrobial peptide of the sequence _{d}(KLAKLAK)₂ (SEQ ID NO: 15).

FIG. 2 shows a bar graph depicting the percent reduction in mitochondrial function of KS cells treated with HK-1 and various other molecules.

FIG. 3 shows a bar graph depicting viability of KS cells cells treated with HK-1 and various other molecules.

### DETAILED DESCRIPTION OF THE INVENTION

The present invention provides a homing conjugate containing an antimicrobial peptide and a tumor homing molecule, wherein the tumor homing molecule comprises a dimer of two endothelium-homing peptide monomers, wherein the conjugate homes to and is internalized by a tumor cell type or tissue comprising angiogenic endothelial cells and exhibits high toxicity thereto, wherein the high toxicity is due to disruption of mitochondrial membranes, and wherein the antimicrobial peptide has low mammalian cell toxicity when not linked to said tumor homing molecule. The present invention is based, in part, on the discovery that dimerization of endothelium-homing peptide monomer confers greatly increased cytotoxic activity on the conjugate.

In a homing conjugate of the invention, the tumor homing molecule portion can include an endothelium-homing peptide that is a dimer consisting of two monomers including, for example, the sequence CNGRC (SEQ ID NO: 1) or a functionally equivalent sequence, and the antimicrobial peptide portion can have an amphipathic α-helical structure such as the sequence _{d}(KLAKLAK)₂ (SEQ ID NO: 15) or the sequence _{d}(ALLLAIRRR) (SEQ ID NO: 7) or the sequence _{d}(ALLLAIRRKKK) (SEQ ID NO: 19). The all D-enantiomer can be used to avoid degradation by proteases (Bessalle et al., FEBS Lett. 274:151-155 (1990); Wade et al., Proc. Natl. Acad. Sci. 87:4761-4765 (1990)).

In a homing conjugate of the invention, the tumor homing molecule portion can include an endothelium-homing peptide that is a dimer consisting of two monomers including, for example, the sequence referred to as “RGD4C,” which is ACDCRGDCFCG (SEQ ID NO: 3) or a functionally equivalent sequence, and the antimicrobial peptide portion can have an amphipathic α-helical structure such as the sequence _{d}(KLAKLAK)₂ (SEQ ID NO: 15) or the sequence _{d}(ALLLAIRRR) (SEQ ID NO: 18) or the sequence _{d}(ALLLAIRRRKKK) (SEQ ID NO: 19).

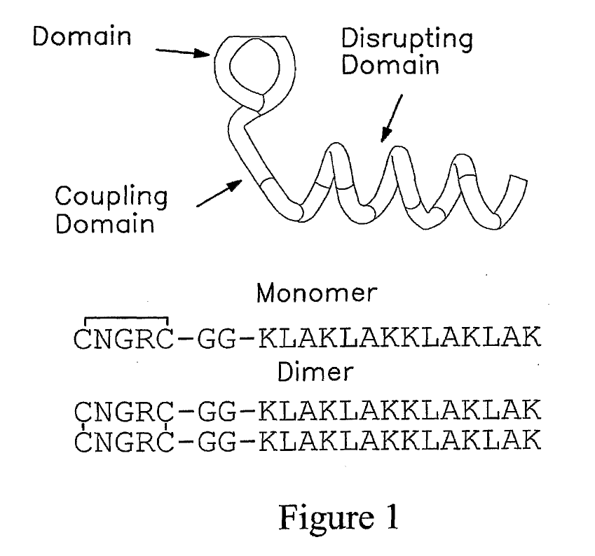

In a preferred embodiment, the antimicrobial peptide portion contains the sequence _{d}(KLALAK)₂ (SEQ ID NO: 15). An exemplary homing conjugate containing an antimicrobial peptide and a tumor homing molecule that includes a dimer of two endothelium-homing peptide monomers is provided herein as (CNGRC-GG-_{d}(KLAKLAK)₂)₂ (SEQ ID NO: 9), which is shown in FIG. 1. A further exemplary homing conjugate containing an antimicrobial peptide and a tumor homing molecule that includes a dimer of two endothelium-homing peptide monomers is provided herein as (ACDCRGDCFCG-GG-_{d}(KLAKLAK)₂)₂ (SEQ ID NO: 12).

A further example of a homing conjugate of the invention containing an antimicrobial peptide and a tumor homing molecule that includes a dimer of two endothelium-homing peptide monomers is provided herein as (CNGRC-GG-_{d}(ALLLAIRRR))₂ (SEQ ID NO: 10). A further example of a homing conjugate of the invention containing an antimicrobial peptide and a tumor homing molecule that includes a dimer of two endothelium-homing peptide monomers is provided herein as (CNGRC-GG-_{d}(ALLLAIRRRKKK))₂ (SEQ ID NO: 11).

The present invention further provides a method of directing an antimicrobial peptide to an angiogenic endothelial tissue or cell type in vivo. The method includes the step of administering a homing conjugate containing an antimicrobial peptide and a tumor homing molecule that includes a dimer of two endothelium-homing peptide monomers at least one of which is linked to an antimicrobial peptide, where the homing conjugate is selectively internalized by angiogenic endothelial tissue and exhibits high toxicity thereto, while the antimicrobial peptide has low mammalian cell toxicity when not linked to the endothelium-homing peptide. In a method of the invention, the endothelium-homing peptide can contain, for example, the sequence CNGRC (SEQ ID NO: 1) or a functionally equivalent sequence, and the antimicrobial peptide can contain a sequence such as (KLAKLAK)₂ (SEQ ID NO: 6). In a preferred embodiment, the homing conjugate contains the sequence (CNGRC-GG-_{d}(KLAKLAK)₂)₂ (SEQ ID NO: 9). The homing conjugate set forth as SEQ ID NO: 3 is shown in FIG. 1 and contains two antimicrobial peptides and a tumor homing molecule that includes a dimer of two endothelium-homing peptide monomers, each linked to an antimicrobial peptide of the sequence KLAKLAK (SEQ ID NO: 5). The all D-enantiomer can be used to avoid degradation by proteases (Bessalle et al., FEBS Lett. 274:151-155 (1990); Wade et al., Proc. Natl. Acad. Sci. 87:4761-4765 (1990)).

In a method of the invention, the endothelium-homing peptide also can contain, for example, the sequence ACDCRGDCFCG (SEQ ID NO: 3) also referred to as “RGD4C” or a functionally equivalent sequence, and the antimicrobial peptide can contain a sequence such as (KLAKLAK)₂ (SEQ ID NO: 6). In a preferred embodiment, the homing conjugate contains the sequence (ACDCRGDCFCG-GG-_{d}(KLAKLAK)₂)₂ (SEQ ID NO: 12). The homing conjugate contains two antimicrobial peptides and a tumor homing molecule that includes a dimer of two endothelium-homing peptide monomers, each linked to an antimicrobial peptide of the sequence KLAKKLAK.

In a further embodiment of the invention, the homing conjugate contains two antimicrobial peptides and a tumor homing molecule that includes a dimer of two endothelium-homing peptide monomers, each linked to an antimicrobial peptide of the sequence ALLLAIRRR. A homing conjugate can contain an antimicrobial peptide and a tumor homing molecule that includes a dimer of two endothelium-homing peptide monomers is provided herein as (ACDCRGDCFCG-GG-_{d}(ALLLAIRRR))₂ (SEQ ID NO: 13) or (CNRGC-GG-_{d}(ALLLAIRRR))₂ (SEQ ID NO: 10).

In a further embodiment of the invention, the homing conjugate contains two antimicrobial peptides and a tumor homing molecule that includes a dimer of two endothelium-homing peptide monomers, each linked to an antimicrobial peptide of the sequence ALLLAIRRRKKK (SEQ ID NO: 8). An example of a homing conjugate of the invention contains an antimicrobial peptide and a tumor homing molecule that includes a dimer of two endothelium-homing peptide monomers is provided herein as (ACDCRGDCFCG-GG-_{d}(ALLLAIRRRKKK))₂ (SEQ ID NO: 14) or (CNRGC-GG-_{d}(ALLLAIRRRKKK)₂ (SEQ ID NO: 11).

Also provided by the invention is a method of inducing selective toxicity in vivo in an angiogenic endothelial cell type or tissue associated with a tumor. The method includes the step of administering to a subject having cancer a chimeric endothelium-homing pro-apoptotic peptide that contains a endothelium-homing peptide linked to an antimicrobial peptide, where the homing conjugate is selectively internalized by an angiogenic endothelial tissue or cell type and exhibits high toxicity thereto, while the antimicrobial peptide has low mammalian cell toxicity when not linked to the endothelium-homing peptide. The method of inducing selective toxicity in an angiogenic endothelial cell type or tissue in vivo can be practiced, for example, with an endothelium-homing molecule that is a dimer of two endothelium-homing peptide monomers, each containing the sequence CNGRC (SEQ ID NO: 1) or a functionally equivalent sequence. The antimicrobial peptide can include, for example, the sequence _{d}(KLAKLAK)₂ (SEQ ID NO: 15) or _{d}(ALLLAIRRRR) (SEQ ID NO: 18) or _{d}(ALLLAIRRRRKKK) (SEQ ID NO: 19). In a preferred embodiment, the homing conjugate includes the sequence (CNGRC-GG-_{d}(KLAKLAK)₂)₂ (SEQ ID NO: 9). In a further embodiment, the homing conjugate includes the sequence (CNGRC-GG-_{d}(ALLLAIRRRR))₂ (SEQ ID NO: 10). In a further embodiment, the homing conjugate includes the sequence (CNGRC-GG-_{d}(ALLLAIRRRRKKK))₂ (SEQ ID NO: 11).

In addition, the invention provides a method of treating a patient having cancer by administering to the patient a homing conjugate of the invention, whereby the homing conjugate is selectively toxic to the tumor. The homing conjugate contains a endothelium-homing molecule that is a dimer of two endothelium-homing peptide monomers, each containing the sequence CNGRC (SEQ ID NO: 1), at least one of which linked to an antimicrobial peptide, and the homing conjugate is selectively internalized by angiogenic endothelial tissue and exhibits high toxicity thereto, while the antimicrobial peptide has low mammalian cell toxicity when not linked to the endothelium-homing peptide. The endothelium-homing peptide portion can contain, for example, the sequence CNGRC (SEQ ID NO: 1) or a functionally equivalent sequence, and the antimicrobial peptide portion can contain, for example, the sequence _{d}(KLAKLAK)₂ (SEQ ID NO: 15) or _{d}(ALLLAIRRRR) (SEQ ID NO: 18). In a preferred embodiment, the homing conjugate includes the sequence (CNGRC-GG-_{d}(KLAKLAK)₂)₂ (SEQ ID NO: 9). In a further embodiment, the homing conjugate includes the sequence (CNGRC-GG-_{d}(ALLLAIRRRR))₂.

Antimicrobial peptides, also known as lytic peptides or channel-forming peptides, are broad spectrum anti-bacterial agents. These peptides typically disrupt bacterial cell membranes, causing cell lysis and death. Over 100 antimicrobial peptides occur naturally. In addition, analogs have been synthesized de novo as described in Javadpour et al., J. Med. Chem. 39:3107-3113 (1996); and Blondelle and Houghten, Biochem. 31: 12688-12694 (1992), each of which is incorporated herein by reference. While some antimicrobial peptides such as melittin are not selective and damage normal mammalian cells at the minimum bactericidal concentration, others are selective for bacterial cells. For example, the naturally occurring magainins and cecropins exhibit substantial bactericidal activity at concentrations that are not lethal to normal mammalian cells.

Antimicrobial peptides frequently contain cationic amino acids, which are attracted to the head groups of anionic phospholipids, leading to the preferential disruption of negatively charged membranes. Once electrostatically bound, the amphipathic helices can distort the lipid matrix, resulting in loss of membrane barrier function (Epand, The Amphipathic Helix CRC Press: Boca Raton (1993); Lugtenberg and van Alphen, Biochim. Biophys. Acta 737:51-115 (1983), each of which is incorporated herein by reference). Prokaryotic cytoplasmic membranes maintain large transmembrane potentials and have a high content of anionic phospholipids. In contrast, the outer leaflet of eukaryotic plasma membranes generally has low, or no, membrane potential and is almost exclusively composed of zwitterionic phospholipids. Thus, due to distinct membrane compositions, antimicrobial peptides can preferentially disrupt prokaryotic membranes as compared to eukaryotic membranes.

The present invention is directed to the surprising discovery that a homing conjugate that includes a dimer of two endothelium-homing peptide monomers, at least one of which is linked to an antimicrobial peptide sequence has greatly increased pro-apoptotic activity compared to a monomeric homing conjugate. A homing conjugate of the invention generally is non-toxic outside of eukaryotic cells, but promotes disruption of mitochondrial membranes and subsequent cell death when targeted and internalized by eukaryotic cells. Homing pro-apoptotic conjugates such as (CNGRC-GG-_{d}(KLAKLAK)₂)₂ (SEQ ID NO: 9), which contains the two copies of the antimicrobial peptide _{d}(KLAKLAK)₂ (SEQ ID NO: 15), each linked to one monomer of the dimeric endothelium homing molecule (CNGRC)₂ (SEQ ID NO: 2), can have selective toxicity against angiogenic endothelial cells in vivo and, thus, be useful as a new class of anti-cancer therapeutics. In addition, homing pro-apoptotic conjugates such as (CNGRC-GG-_{d}(ALLLAIRRR))₂ (SEQ ID NO: 10), which contains the two copies of the antimicrobial peptide _{d}(ALLLAIRRR) (SEQ ID NO: 18), each linked to one monomer of the dimeric endothelium homing molecule (CNGRC)₂ (SEQ ID NO: 2), can have selective toxicity against angiogenic endothelial cells in vivo and, thus, be useful as a new class of anti-cancer therapeutics. Furthermore, homing pro-apoptotic conjugates such as (CNGRC-GG-_{d}(ALLLAIRRRKKK))₂ (SEQ ID NO: 11), which contains the two copies of the antimicrobial peptide _{d}(ALLLAIRRRKKK) (SEQ ID NO: 19), each linked to one monomer of the dimeric endothelium homing molecule (CNGRC)₂ (SEQ ID NO: 2), can have selective toxicity against angiogenic endothelial cells in vivo and, thus, be useful as a new class of anti-cancer therapeutics.

Homing pro-apoptotic conjugates such as (ACDCRGDCFCG-GG-_{d}(KLAKLAK)₂)₂ (SEQ ID NO: 12), which contains the two copies of the antimicrobial peptide _{d}(KLAKLAK)₂ (SEQ ID NO: 15), each linked to one monomer of the dimeric endothelium homing molecule (ACDCRGDCFCG)₂ (SEQ ID NO: 4), can have selective toxicity against angiogenic endothelial cells in vivo and, thus, be useful as a new class of anti-cancer therapeutics. In addition, homing pro-apoptotic conjugates such as (ACDCRGDCFCG-GG-_{d}(ALLLAIRRR))₂ (SEQ ID NO: 13), which contains the two copies of the antimicrobial peptide _{d}(ALLLAIRRR) (SEQ ID NO: 18), each linked to one monomer of the dimeric endothelium homing molecule (ACDCRGDCFCG)₂ (SEQ ID NO: 4), can have selective toxicity against angiogenic endothelial cells in vivo and, thus, be useful as a new class of anti-cancer therapeutics. Furthermore, homing pro-apoptotic conjugates such as (ACDCRGDCFCG-GG-_{d}(ALLLAIRRRKKK)₂ (SEQ ID NO: 14), which contains the two copies of the antimicrobial peptide _{d}(ALLLAIRRRKKK) (SEQ ID NO: 8), each linked to one monomer of the dimeric endothelium homing molecule (ACDCRGDCFCG)₂ (SEQ ID NO: 4), can have selective toxicity against angiogenic endothelial cells in vivo and, thus, be useful as a new class of anti-cancer therapeutics.

Thus, the present invention provides a homing conjugate, which includes a tumor homing molecule containing two tumor homing peptide monomers that selectively home to an angiogenic endothelial cell type or tissue, at least one of the monomers linked to an antimicrobial peptide, where the conjugate is selectively internalized by the angiogenic endothelial cell type or tissue and exhibits high toxicity thereto, and where the antimicrobial peptide has low mammalian cell toxicity when not linked to the tumor homing molecule. A homing pro-apoptotic conjugate of the invention can exhibit selective toxicity against angiogenic endothelial cells and can be useful, for example, in methods of inducing selective toxicity in vivo in a tumor having angiogenic vasculature.

As disclosed herein, a homing conjugate of the invention contains an antimicrobial peptide with selective toxicity against bacteria as compared to eukaryotic cells, and can induce mitochondrial swelling at concentrations significantly less than the concentration required to kill eukaryotic cells such that mitochondrial membranes are preferentially disrupted as compared to eukaryotic membranes. An antimicrobial peptide such as _{d}(KLAKLAK)₂ (SEQ ID NO: 15) can disrupt mitochondrial membranes, which, like bacterial membranes, have a high content of anionic phospholipids, reflecting the common ancestry of bacteria and mitochondria (Epand, supra, 1993; Lugtenberg and van Alphen, supra, 1983; Matsuzaki et al., Biochemistry 34:6521-6526 (1995); Hovius et al., FEBS Lett. 330:71-76 (1993); and Baltcheffsky and Baltcheffsky in Lee et al., Mitochondria and Microsomes Addison-Wesley: Reading, Mass. (1981), each of which is incorporated herein by reference).

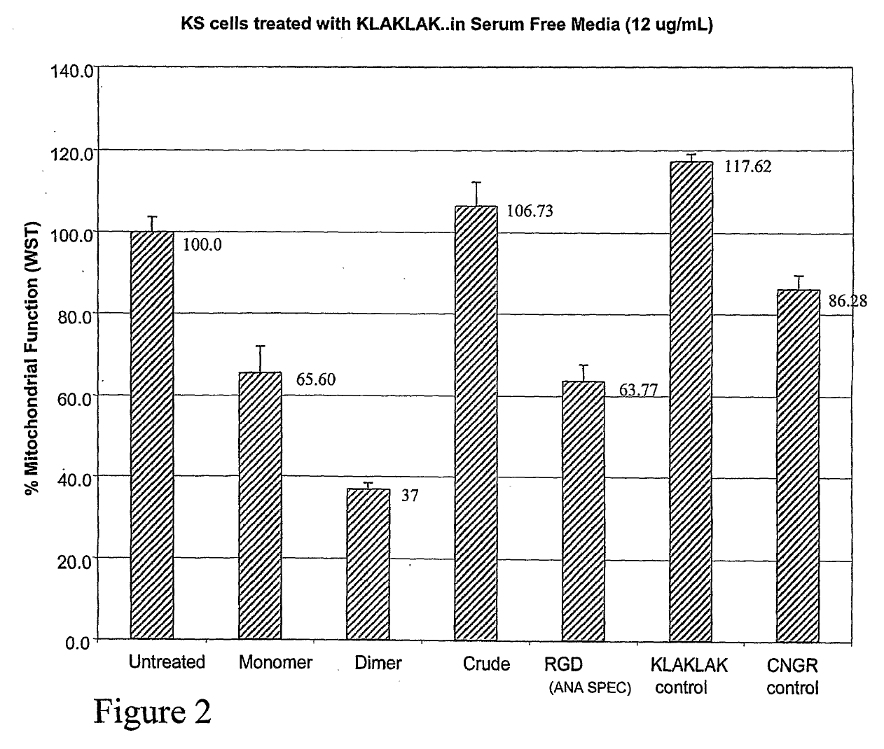

As further disclosed herein, two copies of the antimicrobial peptide _{d}(KLAKLAK)₂ (SEQ ID NO: 15) were conjugated to the tumor homing molecule (CNGRC)₂ (SEQ ID NO: 2) as depicted in FIG. 1. In particular, each copy of the antimicrobial peptide _{d}(KLAKLAK)₂ (SEQ ID NO: 15) was linked to one of the CNGRC (SEQ ID NO: 1) homing peptide monomers via a glycinylglycine bridge to produce the homing conjugate (CNGRC-GG-_{d}(KLAKLAK)₂)₂ (SEQ ID NO: 9) to produce the homing conjugate Hunter-Killer-1 or HK-1. As disclosed herein, HK-1 was tested in a tissue culture model of Kaposi's Sarcoma (KS). In particular, as shown in FIG. 2, treatment with (CNGRC-GG _{d}(KLAKLAK)₂)₂ (SEQ ID NO: 9) (HK-1) resulted in almost 50 percent cell death among KS, as compared to approximately 10 percent cell death upon treatment with a monomeric CNGRC-GG-_{d}(KLAKLAK) (SEQ ID NO: 20) homing conjugate under equivalent conditions.

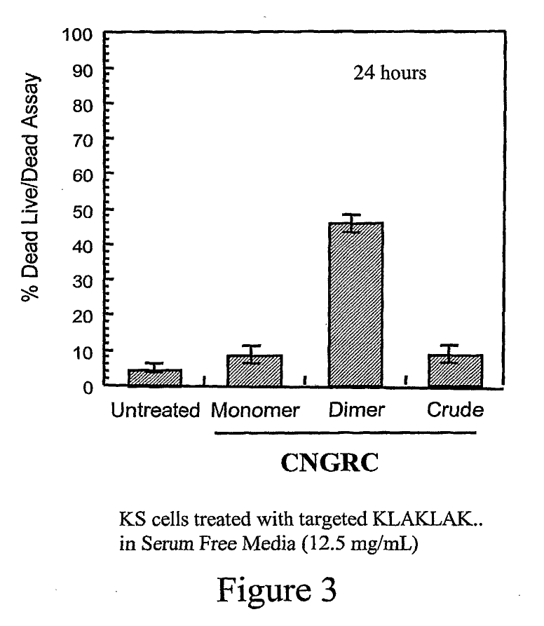

As shown in Example 1, the HK-1 homing conjugate (CNGRC-GG-_{d}(KLAKLAK)₂)₂ (SEQ ID NO: 9) results in a significant reduction in mitochondrial function in treated KS cells. As disclosed in FIG. 3, mitochondrial function in HK-1 treated KS cell culture was reduced to 37 percent as compared to over 65 percent in KS cells treated with a monomeric CNGRC-GG-_{d}(KLAKLAK)₂ (SEQ ID NO: 20) homing conjugate under equivalent conditions.

In sum, these results indicate that homing conjugates containing a dimer consisting of two homing peptide monomers, when linked to one or more antimicrobial peptide sequences can promote disruption of mitochondrial membranes and subsequent cell death when internalized by the targeted eukaryotic target cells, for example, angiogenic endothelial cells. Homing conjugates such as HK-1, which have selective toxicity against angiogenic endothelial cells, can be particularly valuable as anti-cancer therapeutics. A homing conjugate containing a dimer consisting of two homing peptide monomers can contain a tumor homing molecule, or can contain another dimeric homing molecule that selectively homes to a selected mammalian cell type or tissue.

A homing pro-apoptotic conjugate of the invention is characterized by being highly toxic to the mammalian cell type in which it is internalized. As used herein, the term “highly toxic” means that the conjugate is relatively effective in resulting in cell death of a selected cell type or tissue. One skilled in the art understands that toxicity can be analyzed using one of a variety of well known assays for cell viability. In general, the term highly toxic is used to refer to a conjugate in which the concentration for half maximal killing (LC50) is less than about 100 μM, preferably less than about 50 μM.

As used herein, the term “antimicrobial peptide” means a naturally occurring or synthetic peptide having antimicrobial activity, which is the ability to kill or slow the growth of one or more microbes. An antimicrobial peptide can, for example, kill or slow the growth of one or more strains of bacteria including a Gram-positive or Gram-negative bacteria, or a fungi or protozoa. Thus, an antimicrobial peptide can have, for example, bacteriostatic or bacteriocidal activity against, for example, one or more strains of Escherichia coli, Pseudomonas aeruginosa or Staphylococcus aureus. While not wishing to be bound by the following, an antimicrobial peptide can have biological activity due to the ability to form ion channels through membrane bilayers as a consequence of self-aggregation.

An antimicrobial peptide is typically highly basic and can have a linear or cyclic structure. As discussed further below, an antimicrobial peptide can have an amphipathic α-helical structure (see U.S. Pat. No. 5,789,542; Javadpour et al., supra, 1996; Blondelle and Houghten, supra, 1992). An antimicrobial peptide also can be, for example, a β-strand/sheet-forming peptide as described in Mancheno et al., J. Peptide Res. 51:142-148 (1998).

An antimicrobial peptide can be a naturally occurring or synthetic peptide. Naturally occurring antimicrobial peptides have been isolated from biological sources such as bacteria, insects, amphibians and manmals and are thought to represent inducible defense proteins that can protect the host organism from bacterial infection. Naturally occurring antimicrobial peptides include the gramicidins, magainins, mellitins, defensins and cecropins (see, for example, Maloy and Kari, Biopolymers 37:105-122 (1995); Alvarez-Bravo et al., Biochem. J. 302:535-538 (1994); Bessalle et al., FEBS 274:151-155 (1990); and Blondelle and Hougliten in Bristol (Ed.), Annual Reports in Medicinal Chemistry pages 159-168 Academic Press, San Diego, each of which is herein incorporated by reference). As discussed further below, an antimicrobial peptide also can be an analog of a natural peptide, especially one that retains or enhances amphipathicity.

An antimicrobial peptide incorporated within a homing pro-apoptotic conjugate of the invention has low mammalian cell toxicity when not linked to a tumor homing molecule. Mammalian cell toxicity readily can be assessed using routine assays. For example, mammalian cell toxicity can be assayed by lysis of human erythrocytes in vitro as described in Javadpour et al., supra, 1996. An antimicrobial peptide having “low mammalian cell toxicity” is not lytic to human erythrocytes or requires concentrations of greater than 100 μM for lytic activity, preferably concentrations greater than 200, 300, 500 or 1000 μM.

In a preferred embodiment, the invention provides a homing conjugate in which the antimicrobial peptide portion promotes disruption of mitochondrial membranes when internalized by eukaryotic cells. In particular, such an antimicrobial peptide preferentially disrupts mitochondrial membranes as compared to eukaryotic membranes. Mitochondrial membranes, like bacterial membranes but in contrast to eukaryotic plasma membranes, have a high content of negatively charged phospholipids. An antimicrobial peptide can be assayed for activity in disrupting mitochondrial membranes using, for example, an assay for initochondrial swelling as described in U.S. patent application Ser. No. 09/765,086; published Nov. 29, 2001 as Publication No. 20010046498, or another assay well known in the art. As disclosed U.S. patent application Ser. No. 09/765,086, for example, _{d}(KLAKLAK)₂ induced marked mitochondrial swelling at a concentration of 10 μM, significantly less than the concentration required to kill eukaryotic cells. An antimicrobial peptide that induces significant mitochondrial swelling at, for example, 50 μM, 40 μM, 30 μM, 20 μM, 10 μM, or less, is considered a peptide that promotes disruption of mitochondrial membranes.

The invention also provides a homing conjugate encompassing a tumor homing molecule containing a dimer consisting of two homing peptide monomers, each linked to an antimicrobial peptide having an amphipathic α-helical structure. In a homing conjugate of the invention, the antimicrobial peptide portion can have, for example, the sequence _{d}(KLAKLAK)₂ (SEQ ID NO: 15) or the sequence _{d}(ALLLAIRRR) (SEQ ID NO: 18) or the sequence _{d}(ALLLAIRRRKKK)₂.

Antimicrobial peptides generally have random coil conformations in dilute aqueous solutions, yet high levels of helicity can be induced by helix-promoting solvents and amphipathic media such as micelles, synthetic bilayers or cell membranes. α-Helical structures are well known in the art, with an ideal α-helix characterized by having 3.6 residues per turn and a translation of 1.5 per residue (5.4 per turn; see Creighton, Proteins: Structures and Molecular Properties W.H Freeman, New York (1984)). In an amphipathic α-helical structure, polar and non-polar amino acid residues are aligned into an amphipathic helix, which is an α-helix in which the hydrophobic amino acid residues are predominantly on one face, with hydrophilic residues predominantly on the opposite face when the peptide is viewed along the helical axis.

Antimicrobial peptides of widely varying sequence have been isolated, sharing an amphipathic α-helical structure as a common feature (Saberwal et al., Biochim. Biophys. Acta 1197:109-131 (1994)). Analogs of native peptides with amino acid substitutions predicted to enhance amphipathicity and helicity typically have increased antimicrobial activity. In general, analogs with increased antimicrobial activity also have increased cytotoxicity against mammalian cells (Maloy et al., Biopolymers 37:105-122 (1995)).

As used herein in reference to an antimicrobial peptide, an amphipathic α-helical structure refers an α-helix with a hydrophilic face containing several polar residues at physiological pH and a hydrophobic face containing nonpolar residues. A polar residue can be, for example, a lysine or arginine residue, while a nonpolar residue can be, for example, a leucine or alanine residue. An antimicrobial peptide having an amphipathic α-helical structure generally has an equivalent number of polar and nonpolar residues within the amphipathic domain and a sufficient number of basic residues to give the peptide an overall positive charge at neutral pH (Saberwal et al., Biochim. Biophys. Acta 1197:109-131 (1994), which is incorporated by reference herein). One skilled in the art understands that helix-promoting amino acids such as leucine and alanine can be advantageously included in an antimicrobial peptide of the invention (see, for example, Creighton, supra, 1984).

A variety of antimicrobial peptides having an amphipathic α-helical structure are well known in the art. Such peptides include synthetic, minimalist peptides based on a heptad building block scheme in which repetitive heptads are composed of repetitive trimers with an additional residue. Such synthetic antimicrobial peptides include, for example, peptides of the general formula [(X1X2X2)(X1X2X2)X1]n or [(X1X2X2)X1(X1X2X2)]n, where X1 is a polar residue, X2 is a nonpolar residue; and n is 2 or 3 (see Javadpour et al., supra, 1996). _{d}(KLAKLAK)₂ (SEQ ID NO: 15); _{d}(KLAKKLA)₂ (SEQ ID NO: 21); _{d}(KAAKKAA)₂ (SEQ ID NO: 16); and _{d}(KLGKKLG)₂ (SEQ ID NO: 17) are examples of synthetic antimicrobial peptides having an amphipathic α-helical structure. Similar synthetic, antimicrobial peptides having an amphipathic α-helical structure also are known in the art, for example, as described in U.S. Pat. No. 5,789,542 to McLaughlin and Becker.

Helicity readily can be determined by one skilled in the art, for example, using circular dichroism spectroscopy. Percent α-helicity can be determined, for example, after measuring molar ellipticity at 222 nm as described in Javadpour et al., supra, 1996 (see, also, McLean et al., Biochemistry 30:31-37 (1991), which is incorporated by reference herein). An amphipathic α-helical antimicrobial peptide of the invention can have, for example, at least about 20% helicity when assayed in amphipathic media such as 25 mMSDS. One skilled in the art understands that such an antimicrobial peptide having an amphipathic α-helical structure can have, for example, at least about 25%, 30%, 35% or 40% helicity when assayed in 25 mM SDS. An antimicrobial peptide having an α-helical structure can have, for example, from 25% to 90% helicity; 25% to 60% helicity; 25% to 50% helicity; 25% to 40% helicity; 30% to 90% helicity; 30% to 60% helicity; 30% to 50% helicity; 40% to 90% helicity or 40% to 60% helicity when in assayed in 25 mM SDS. Amphipathicity can readily be determined, for example, using a helical wheel representation of the peptide (see, for example, Blondelle and Houghten, supra, 1994).

The structure of an exemplary homing conjugate of the invention, (CNGRC-GG-_{d}(KLAKLAK)₂)₂ (SEQ ID NO: 9) is illustrated in FIG. 1. As can be seen in FIG. 1, the homing domain, (CNGRC)₂ (SEQ ID NO: 2) is a dimer of two disulfide-bonded CNGRC monomers, each of which is in turn coupled to a membrane disrupting domain, (KLAKLAKKLAKLAK) (SEQ ID NO: 6) via a glycinylglycine bridge. Furthermore, the membrane disrupting (KLAKLAKKLAKLAK) (SEQ ID NO: 6) portion forms an amphipathic helix. In particular, the lysine residues are aligned on one face of the helix (shown as dark shaded region of helix), while the non-polar leucine and alanine residues are aligned on the opposite (light-shaded) face of the helix.

A homing conjugate of the invention can be a homing conjugate in which the tumor homing molecule is a dimer consisting of two tumor homing peptide monomers. A homing conjugate of the invention can have a variety of sizes, from about 36 amino acids to about fifty amino acids or more. A homing conjugate of the invention can have, for example, from about 20 to about 70 amino acids, preferably from 20 to 50 amino acids, more preferably from 30 to 40 amino acids. Such a homing conjugate can have, for example, an upper length of 75, 70, 65, 60, 50, 40, 36, 35, 30, 27, 25 or 21 amino acids. A homing conjugate of the invention can be linear or cyclic. In a preferred embodiment, a homing pro-apoptotic homing conjugate of the invention includes a dimer consisting of two tumor homing peptide monomers.

A homing conjugate of the invention also can be a peptidomimetic and corresponding peptidomimetics are included within the homing conjugates of the invention. As used herein, the term peptidomimetic is used broadly to mean a peptide-like molecule that has substantially the activity of the corresponding peptide. Peptidomimetics include chemically modified peptides, peptide-like molecules containing non-naturally occurring amino acids, peptoids and the like, have the selective homing activity and the high toxicity of the peptide from which the peptidomimetic is derived (see, for example, “Burger's Medicinal Chemistry and Drug Discovery” 5th ed., vols. 1 to 3 (ed. M. E. Wolff; Wiley Interscience 1995), which is incorporated herein by reference). For example, D amino acids can be advantageously included in the antimicrobial peptide portion of a homing conjugate of the invention. Peptidomimetics provide various advantages over a peptide, including increased stability during passage through the digestive tract and, therefore, can be advantageously used as oral therapeutics.

In a homing pro-apoptotic conjugate of the invention, a coupling domain can be used to link a tumor homing peptide and an antimicrobial peptide and can, for example, impart flexibility to the conjugate as a whole. A coupling domain can be, for example, a glycinylglycine linker, alaninylalanine linker or other linker incorporating glycine, alanine or other amino acids.

The vasculature within a tumor generally undergoes active angiogenesis, resulting in the continual formation of new blood vessels to support the growing tumor. Such angiogenic blood vessels are distinguishable from mature vasculature in that angiogenic vasculature expresses unique endothelial cell surface markers, including the αvβ3 integrin (Brooks, Cell 79:1157-1164 (1994); WO 95/14714, Int. Filing Date Nov. 22, 1994) and receptors for angiogenic growth factors (Mustonen and Alitalo, J. Cell Biol. 129:895-898 (1995); Lappi, Semin. Cancer Biol. 6:279-288 (1995)). Many human tumors express this integrin, which may be involved in the progression of certain tumors such as malignant melanomas (Albelda et al., Cancer Res. 50:6757-6764 (1990); Danen et al., Int. J. Cancer 61:491-496 (1995); Felding-Habermann et al., J. Clin. Invest. 89:2018-2022 (1992); Sanders et al., Cold Spring Harb. Symp. Quant. Biol. 58:233-240 (1992); Mitjans et al., J. Cell. Sci. 108:3067-3078 (1995)). Moreover, tumor vasculature is histologically distinguishable from other blood vessels in that tumor vasculature is fenestrated (Folkman, Nature Med. 1:27-31 (1995); Rak et al., Anticancer Drugs 6:3-18 (1995)). Thus, the unique characteristics of tumor vasculature make it a particularly attractive target for anti-cancer therapeutics.

As disclosed herein, tumor homing molecules can bind to the endothelial lining of small blood vessels of tumors. The vasculature within tumors is distinct, presumably due to the continual neovascularization, resulting in the formation of new blood vessels required for tumor growth. The distinct properties of the angiogenic neovasculature within tumors are reflected in the presence of specific markers in endothelial cells and pericytes (Folkman, Nature Biotechnol. 15:510 (1997); Risau, FASEB J. 9:926-933 (1995); Brooks et al., supra, 1994); these markers likely are being targeted by the disclosed tumor homing molecules.

The ability of a tumor homing molecule to target the blood vessels in a tumor provides substantial advantages over methods of systemic treatment or methods that directly target the tumor cells. For example, tumor cells depend on a vascular supply for survival and the endothelial lining of blood vessels is readily accessible to a circulating probe. Conversely, in order to reach solid tumor cells, a therapeutic agent must overcome potentially long diffusion distances, closely packed tumor cells, and a dense fibrous stroma with a high interstitial pressure that impedes extravasation (Burrows and Thorpe, Pharmacol. Ther. 64:155-174 (1994)).

In addition, where the tumor vasculature is targeted, the killing of all target cells may not be required, since partial denudation of the endothelium can lead to the formation of an occlusive thrombus halting the blood flow through the entirety of the affected tumor vessel (Burrows and Thorpe, supra, 1994). Furthermore, unlike direct tumor targeting, there is an intrinsic amplification mechanism in tumor vasculature targeting. A single capillary loop can supply nutrients to up to 100 tumor cells, each of which is critically dependent on the blood supply (Denekamp, Cancer Metast. Rev. 9:267-282 (1990); Folkman, supra, 1997).

As set forth above and exemplified herein, a tumor homing molecule that is selective for the angiogenic endothelial cells of tumor vasculature can be particularly useful for directing an antimicrobial peptide to tumor vasculature, while reducing the likelihood that the antimicrobial peptide will have a toxic effect on normal, healthy organs or tissues. Thus, in one embodiment, the invention provides a homing conjugate, which includes a tumor homing molecule containing a dimer consisting of two homing peptide monomers that selectively homes to angiogenic endothelial cells, each of the monomers linked to an antimicrobial peptide, where the conjugate is selectively internalized by angiogenic endothelial cells and exhibits high toxicity thereto, and where the antimicrobial peptide has low mammalian cell toxicity when not linked to the tumor homing molecule.

As used herein, the term “selective toxicity” means enhanced cell death in a selected cell type or tissue as compared to a control cell type or tissue. In general, selective toxicity is characterized by at least a two-fold greater extent of cell death in the selected cell type or tissue, such as angiogenic endothelial cells, as compared to a control cell type or tissue, for example, angiostatic endothelial cells. Thus, as used herein, the term selective toxicity encompasses specific toxicity, whereby cell death occurs essentially only the selected cell type or tissue, as well as toxicity occurring in a limited number of cell types or tissues in addition to the selected cell type or tissue. One skilled in the art further understands that the term selective toxicity refers to cell death effected by all mechanisms including apoptotic and necrotic cell death. Thus, a homing conjugate of the invention that exhibits selective toxicity for angiogenic endothelial cells effects enhanced cell death of the angiogenic endothelial cells as compared to angiostatic endothelial cells or surrounding cells of other types.

As disclosed herein, identified tumor homing molecules containing a dimer consisting of two homing peptide monomers are useful for targeting a desired antimicrobial peptide, which is linked to the homing molecule, to a selected cell type such as angiogenic endothelial cells. After being internalized by the angiogenic endothelial cells in tumor vasculature, the antimicrobial peptide is toxic to the endothelial cells, thereby restricting the blood supply to the tumor and inhibiting tumor growth.

A tumor homing molecule useful in the homing pro-apoptotic conjugates of the invention can be a peptide containing, for example, an NGR motif, such as CNGRC (SEQ ID NO: 1). A tumor homing molecule useful in the homing pro-apoptotic conjugates of the invention can be a peptide containing, for example, an RGD motif, such as the RGD4C sequence, which is ACDCRGDCFCG (SEQ ID NO: 3). Tumor homing molecules can be identified by screening a library of molecules by in vivo panning as set forth in U.S. Pat. No. 5,622,699, issued Apr. 22, 1997; and Pasqualini and Ruoslahti, Nature 380:364-366 (1996), each of which is incorporated herein by reference.

The term “tumor homing molecule,” as used herein, means a peptide or peptidomimetic or protein dimer that contains two homing peptide monomers and that selectively homes in vivo to a selected cell type or tissue. By “selectively homes” is meant that, in vivo, the tumor homing molecule binds preferentially to a selected cell type or tissue as compared to a control cell type, tissue or organ and generally is characterized by at least a two-fold greater localization at the selected cell type or tissue compared to a control cell type or tissue. A tumor homing molecule useful in the invention can be, for example, a molecule that binds preferentially to the endothelial cells of angiogenic vasculature as compared to other cell types or angiostatic vasculature.

Tumor homing molecules can identified using in vivo panning as follows. By panning in vivo against a tumor cell type, for example, breast carcinoma, a melanoma, Kaposi's sarcoma, phage expressing various peptides that selectively homed to tumors can be identified. Due to the large size of the phage (900-1000 nm) that is used and the short time the phage is allowed to circulate (3 to 5 min), it is unlikely that a substantial number of phage would have exited the circulatory system, particularly in the brain and kidney. Tissue staining studies can be performed to confirm that a tumor homing molecule primarily homes to and binds endothelial cell surface markers, which likely are expressed in an organ-specific manner. Thus, in vivo panning methods well known in the art can be used to identify and analyze endothelial cell specificities. Such an analysis is not possible using endothelial cells in culture because the cultured cells tend to lose their tissue-specific differences (Pauli and Lee, Lab. Invest. 58:379-387 (1988)). Tumor homing peptides can pass through the blood vessels in the tumor, possibly due to the fenestrated nature of the blood vessels, and can be useful for identifying target molecules expressed by tumor cells, as well as target molecules expressed by endothelial cells.

Phage peptide display libraries useful for identifying tumor homing peptides can be constructed essentially as described in U.S. patent application Ser. No. 09/765,086; Smith and Scott, supra, 1993; see, also, Koivunen et al., Biotechnology 13:265-270 (1995); Koivunen et al., Meth. Enzymol. 245:346-369 (1994b), each of which is incorporated herein by reference). Oligonucleotides encoding peptides having substantially random amino acid sequences can be synthesized based on an “NNK” codon, wherein “N” is A, T, C or G and “K” is G or T. “NNK” encodes 32 triplets, which encode the twenty amino acids and an amber STOP codon (Scott and Smith, supra, 1990. The oligonucleotides can be inserted in frame with the sequence encoding the gene III protein (gIII) in the vector fuse 5 such that a peptide-gIII fusion protein is expressed. Following expression, the fusion protein is expressed on the surface of the phage containing the vector (Koivunen et al., supra, 1994b; Smith and Scott, supra, 1993).

The tumor homing peptide CNGRC, which is a monomer building block of the tumor homong molecule of the homing conjugate NK-1, contains the asparagine-glycine-arginine (NGR) motif, which is a weak integrin binding motif similar to the motifs present in integrin-binding peptides (Ruoslahti et al., U.S. Pat. No. 5,536,814, issued Jul. 16, 1996, which is incorporated herein by reference; see, also, Koivunen et al., supra, 1994a). Additional homing conjugates of the invention can contain tumor homing molecules that encompass a dimer consisting of two tumor homing peptide monomers, in which the tumor homing molecule portion contains an NGR motif, RGD motif or GSL motif, can be used to target a linked antimicrobial peptide to the endothelial cells of angiogenic vasculature.

In one embodiment, the invention provides a homing pro-apoptotic conjugate, which includes a tumor homing peptide containing the sequence NGR linked to an antimicrobial peptide. In such a homing pro-apoptotic conjugate of the invention, the tumor homing peptide can be, for example, CNGRC (SEQ ID NO: 1) or ACDCRGDCFCG (SEQ ID NO: 3). In a preferred embodiment, the homing pro-apoptotic conjugate includes the sequence (CNGRC-GG _{d}(KLAKKLAK)₂)₂ (SEQ ID NO: 9). In additional embodiments the homing pro-apoptotic conjugate includes the sequence (CNGRC-GG _{d}(ALLLAIRRR))₂ (SEQ ID NO: 10) and (CNGRC-GG _{d}(ALLLAIRRRKKK))₂ (SEQ ID NO: 11).

Peptide motifs that are useful in tumor homing peptide monomers that make up a tumor homing molecule dimer can be any motif known or confirmed to bind receptor sites in tumor vasculature as described, for example, in U.S. patent application Ser. No. 09/765,086. Such motifs can include, for example NGR, RGD and GSL. The conserved RGD, NGR and GSL motifs can be useful in tumor homing peptide monomers and, in particular, for forming homing conjugates that can selectively deliver an antimicrobial peptide to a tumor. Thus, a tumor homing peptide monomer can comprise the amino acid sequence RGD or NGR or GSL and can be a peptide as small as five amino acids, such as CNGRC. NGR peptides were able to deliver a therapeutically effective amount of doxorubicin to breast tumors, indicating that, even where a tumor homing molecule homes only to tumor vasculature, i.e., not directly to the tumor cells, such vasculature targeting in sufficient to confer the effect of the moiety linked to the molecule. Such tumor homing peptide monomer also can be not only at least 13 amino acids in length, which is the largest peptide exemplified herein, but can be up to 20 amino acids, or 30 amino acids, or 50 to 100 amino acids in length, as desired. A tumor homing peptide monomer that is part of a tumor homing molecule dimer that is incorporated into a homing conjugate of the invention can be produced by chemical synthesis.

In sum, tumor homing molecules can be identified by in vivo panning methods well known in the art and, in some cases, a tumor homing molecule can home to vascular tissue in the tumor as well as to tumor parenchyma, probably due to the fenestrated nature of the blood vessels permitting ready exit from the circulatory system. Due to the ability of such tumor homing molecules to home to tumors, the molecules are useful for targeting a linked antimicrobial peptide to tumors. Thus, the invention provides conjugates comprising a tumor homing molecule that is a dimer consisting of two tumor homing peptide monomers linked to a moiety, such conjugates being useful for targeting the moiety to tumor cells.

The ability of a molecule that homes to a particular tumor to selectively home to another tumor of the same or a similar histologic type can be determined using, for example, human tumors grown in nude mice or mouse tumors grown in syngeneic mice for these experiments. For example, various human breast cancer cell lines, including MDA-MB-435 breast carcinoma (Price et al., Cancer Res. 50:717-721 (1990)), SKBR-1-II and SK-BR-3 (Fogh et al., J. Natl. Cancer Inst. 59:221-226 (1975)), and mouse mammary tumor lines, including EMT6 (Rosen et al., Int. J. Cancer 57:706-714 (1994)) and C3-L5 (Lala and Parhar, Int. J. Cancer 54:677-684 (1993)), are readily available and commonly used as models for human breast cancer. Using such breast tumor models, for example, information relating to the specificity of an identified breast tumor homing molecule for diverse breast tumors can be obtained and molecules that home to a broad range of different breast tumors or provide the most favorable specificity profiles can be identified. In addition, such analyses can yield new information, for example, about tumor stroma, since stromal cell gene expression, like that of endothelial cells, can be modified by the tumor in ways that cannot be reproduced in vitro.

Selective homing of a molecule such as a peptide or protein to a tumor can be due to specific recognition by the peptide of a particular cell target molecule such as a cell surface receptor present on a cell in the tumor. Selectivity of homing is dependent on the particular target molecule being expressed on only one or a few different cell types, such that the molecule homes primarily to the tumor. As discussed above, the identified tumor homing peptides, at least in part, can recognize endothelial cell surface markers in the blood vessels present in the tumors. However, most cell types, particularly cell types that are unique to an organ or tissue, can express unique target molecules. Thus, in vivo panning can be used to identify molecules that selectively home to a particular type of tumor cell such as a breast cancer cell; specific homing can be demonstrated by performing the appropriate competition experiments.

As used herein, the term “tumor” means a mass of cells that are characterized, at least in part, by containing angiogenic vasculature. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a “cancer,” a tumor also can be nonmalignant, provided that neovascularization is associated with the tumor. The term “normal” or “nontumor” tissue is used to refer to tissue that is not a “tumor.” As disclosed herein, a tumor homing molecule can be identified based on its ability to home a tumor, but not to a corresponding nontumor tissue.

As used herein, the term “corresponding,” when used in reference to tumors or tissues or both, means that two or more tumors, or two or more tissues, or a tumor and a tissue are of the same histologic type. The skilled artisan will recognize that the histologic type of a tissue is a function of the cells comprising the tissue. Thus, the artisan will recognize, for example, that a nontumor tissue corresponding to a breast tumor is normal breast tissue, whereas a nontumor tissue corresponding to a melanoma is skin, which contains melanocytes. Furthermore, for purposes of the invention, it is recognized that a tumor homing molecule can bind specifically to a target molecule expressed by the vasculature in a tumor, which generally contains blood vessels undergoing neovascularization, in which case a tissue corresponding to the tumor would comprise nontumor tissue containing blood vessels that are not undergoing active angiogenesis.

A tumor homing molecule useful in the invention can be identified by screening a library of molecules by in vivo panning as set forth in U.S. patent application Ser. No. 09/765,086; U.S. Pat. No. 5,622,699, issued Apr. 22, 1997; and Pasqualini and Ruoslahti, Nature 380:364-366 (1996), each of which is incorporated herein by reference).

A library can contain a few or a large number of different molecules, varying from about ten molecules to several billion molecules or more. If desired, a molecule can be linked to a tag, which can facilitate recovery or identification of the molecule. As used herein, the term “molecule” is used to mean a polymeric or non-polymeric organic chemical such as a drug; a nucleic acid molecule such as an RNA, a cDNA or an oligonucleotide; a peptide, including a variant or modified peptide or peptide-like molecules, referred to herein as peptidomimetics, which mimic the activity of a peptide; or a protein such as an antibody or a growth factor receptor or a fragment thereof such as an Fv, Fd or Fab fragment of an antibody, which contains a binding domain. For convenience, the term “peptide” is used broadly herein to mean peptides, proteins, fragments of proteins and the like. A molecule also can be a non-naturally occurring molecule, which does not occur in nature, but is produced as a result of in vitro methods, or can be a naturally occurring molecule such as a protein or fragment thereof expressed from a cDNA library.

A tumor homing molecule also can be a peptidomimetic, which means a peptide-like molecule that has the binding activity of the tumor homing peptide. With respect to the tumor homing peptide monomers of the invention, peptidomimetics, which include chemically modified peptides, peptide-like molecules containing non-naturally occurring amino acids, peptoids and the like, have the binding activity of a tumor homing peptide upon which the peptidomimetic is derived (see, for example, “Burger's Medicinal Chemistry and Drug Discovery,” supra, 1995).

Methods for identifying a peptidomimetic are well known in the art and include, for example, the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). This structural depository is continually updated as new crystal structures are determined and can be screened for compounds having suitable shapes, for example, the same shape as a tumor homing molecule, as well as potential geometrical and chemical complementarity to a target molecule bound by a tumor homing peptide. Where no crystal structure of a tumor homing peptide or a target molecule that binds the tumor homing molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of a tumor homing molecule.

Methods for preparing libraries containing diverse populations of various types of molecules such as peptides, peptoids and peptidomimetics are well known in the art and various libraries are commercially available (see, for example, Ecker and Crooke, Biotechnology 13:351-360 (1995), and Blondelle et al., Trends Anal. Chem. 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37:1385-1401 (1994), each of which is incorporated herein by reference). Where a molecule is a peptide, protein or fragment thereof, the molecule can be produced in vitro directly or can be expressed from a nucleic acid, which can be produced in vitro. Methods of synthetic peptide and nucleic acid chemistry are well known in the art.

A library of molecules also can be produced, for example, by constructing a cDNA expression library from mRNA collected from a cell, tissue, organ or organism of interest. Methods for producing such libraries are well known in the art (see, for example, Sambrook et al., Molecular Cloning: A laboratory manual (Cold Spring Harbor Laboratory Press 1989), which is incorporated herein by reference). Preferably, a peptide encoded by the cDNA is expressed on the surface of a cell or a virus containing the cDNA. For example, cDNA can be cloned into a phage vector such as fuse 5, wherein, upon expression, the encoded peptide is expressed as a fusion protein on the surface of the phage.

In addition, a library of molecules can comprise a library of nucleic acid molecules, which can be DNA or RNA or an analog thereof. Nucleic acid molecules that bind, for example, to a cell surface receptor are well known (see, for example, O'Connell et al., Proc. Natl. Acad. Sci., USA 93:5883-5887 (1996); Tuerk and Gold, Science 249:505-510 (1990); Gold et al., Ann. Rev. Biochein. 64:763-797 (1995), each of which is incorporated herein by reference). Thus, a library of nucleic acid molecules can be administered to a subject having a tumor, and tumor homing molecules subsequently identified by in vivo panning. If desired, the nucleic acid molecules can be nucleic acid analogs that, for example, are less susceptible to attack by nucleases (see, for example, Jelinek et al., Biochemistry 34:11363-11372 (1995); Latham et al., Nucl. Acids Res. 22:2817-2822 (1994); Tam et al., Nucl. Acids Res. 22:977-986 (1994); Reed et al., Cancer Res. 59:6565-6570 (1990), each of which is incorporated herein by reference).

As set forth herein, in vivo panning can be used to identify a tumor homing peptide that is useful as monomer in a tumor homing molecule portion of the homong conjugate, and which can be linked to an antimicrobial peptide in a homing conjugate of the invention. In vivo panning comprises administering a library to a subject, collecting a sample of a tumor and identifying a tumor homing peptide. The presence of a tumor homing peptide can be identified using various methods well known in the art. Generally, the presence of a tumor homing peptide in a tumor is identified based on one or more characteristics common to the peptides present in the library, then the structure of a particular tumor homing peptide is identified. For example, a highly sensitive detection method such as mass spectrometry, either alone or in combination with a method such as gas chromatography, can be used to identify tumor homing peptides in a tumor. Thus, where a library comprises diverse molecules based generally on the structure of an organic molecule such as a drug, a tumor homing molecule can be identified by determining the presence of a parent peak for the particular molecule.

If desired, the tumor can be collected, then processed using a method such as HPLC, which can provide a fraction enriched in molecules having a defined range of molecular weights or polar or nonpolar characteristics or the like, depending, for example, on the general characteristics of the peptides comprising the library. Conditions for HPLC will depend on the chemistry of the particular molecule and are well known to those skilled in the art. Similarly, methods for bulk removal of potentially interfering cellular materials such as DNA, RNA, proteins, lipids or carbohydrates are well known in the art, as are methods for enriching a fraction containing an organic molecule using, for example, methods of selective extraction. In addition, a library can comprise a population of diverse peptides, each linked to a common, shared tag. Based on the presence and properties of the shared tag, peptides of the library that selectively home to a tumor can be substantially isolated from a sample of the tumor. These and other methods can be useful for enriching a sample of a collected tumor for the particular tumor homing peptide, thereby removing potentially contaminating materials from the collected tumor sample and increasing the sensitivity of detecting a peptide.

A tumor homing peptide will be present in substantial numbers in a tumor following in vivo homing, thereby increasing the ease with which the homing peptides can be identified. Ease of identification of a tumor homing peptide, particularly an untagged molecule, depends on various factors, including the presence of potentially contaminating background cellular material. Thus, where the tumor homing molecule is an untagged peptide, a larger number must home to the tumor in order to identify the specific peptides against the background of cellular protein. The skilled artisan will recognize that the method of identifying a molecule will depend, in part, on the chemistry of the particular molecule.

The peptides of a library can be tagged, which can facilitate recovery or identification of the molecule. As used herein, the term “tag” means a physical, chemical or biological moiety such as a plastic microbead, an oligonucleotide or a bacteriophage, respectively, that is linked to a molecule of the library. Methods for tagging a molecule are well known in the art (Hermanson, Bioconjugate Techniques (Academic Press 1996), which is incorporated herein by reference).

A tag, which can be a shared tag or a specific tag, can be useful for identifying the presence or structure of a tumor homing peptide of a library. As used herein, the term “shared tag” means a physical, chemical or biological moiety that is common to each molecule in a library. Biotin, for example, can be a shared tag that is linked to each molecule in a library. A shared tag can be useful to identify the presence of a molecule of the library in a sample and also can be useful to substantially isolate the molecules from a sample. For example, where the shared tag is biotin, the biotin-tagged molecules in a library can be substantially isolated by binding to streptavidin, or their presence can be identified by binding with a labeled streptavidin. Where a library is a phage display library, the phage that express the peptides are another example of a shared tag, since each peptide of the library is linked to a phage. In addition, a peptide such as the hemaglutinin antigen can be a shared tag that is linked to each molecule in a library, thereby allowing the use of an antibody specific for the hemaglutinin antigen to substantially isolate molecules of the library from a sample of a selected tumor.

A tag also can be a specific tag, which is a physical, chemical or biological tag that is linked to a particular molecule in a library and is unique for that particular molecule. A specific tag is particularly useful if it is readily identifiable. A nucleotide sequence that is unique for a particular molecule of a library is an example of a specific tag. For example, the method of synthesizing peptides tagged with a unique nucleotide sequence provides a library of molecules, each containing a specific tag, such that upon determining the nucleotide sequence, the identity of the peptide is known (see Brenner and Lerner, Proc. Natl. Acad. Sci., USA 89:5381-5383 (1992), which is incorporated herein by reference). The use of a nucleotide sequence as a specific tag for a peptide or other type of molecule provides a simple means to identify the presence of the molecule in a sample because an extremely sensitive method such as PCR can be used to determine the nucleotide sequence of the specific tag, thereby identifying the sequence of the molecule linked thereto. Similarly, the nucleic acid sequence encoding a peptide expressed on a phage is another example of a specific tag, since sequencing of the specific tag identifies the amino acid sequence of the expressed peptide.

The presence of a shared tag or a specific tag can provide a means to identify or recover a tumor homing peptide following in vivo panning. In addition, the combination of a shared tag and specific tag can be particularly useful for identifying a tumor homing molecule. For example, a library of peptides can be prepared such that each is linked to a specific nucleotide sequence tag (see, for example, Brenner and Lerner, supra, 1992), wherein each specific nucleotide sequence tag has incorporated therein a shared tag such as biotin. Upon homing to a tumor, the particular tumor homing peptides can be substantially isolated from a sample of the tumor based on the shared tag and the specific peptides can be identified, for example, by PCR of the specific tag (see Erlich, supra, 1989).

A tag also can serve as a support, which means a tag having a defined surface to which a molecule can be attached. In general, a tag useful as a support is a shared tag. For example, a support can be a biological tag such as a virus or virus-like particle such as a bacteriophage (“phage”); a bacterium such as E. coli; or a eukaryotic cell such as a yeast, insect or mammalian cell; or can be a physical tag such as a liposome or a microbead, which can be composed of a plastic, agarose, gelatin or other biological or inert material. If desired, a shared tag useful as a support can have linked thereto a specific tag. Thus, a phage display library, for example, can be considered to consist of the phage, which is a shared tag that also is a support, and the nucleic acid sequence encoding the expressed peptide, the nucleic acid sequence being a specific tag.

In general, a support should have a diameter less than about 10 μm to about 50 μm in its shortest dimension, such that the support can pass relatively unhindered through the capillary beds present in the subject and not occlude circulation. In addition, a support can be nontoxic, so that it does not perturb the normal expression of cell surface molecules or normal physiology of the subject, and biodegradable, particularly where the subject used for in vivo panning is not sacrificed to collect a selected tumor.

Where a peptide is linked to a support, the tagged molecule comprises the molecule attached to the surface of the support, such that the part of the molecule suspected of being able to interact with a target molecule in a cell in the subject is positioned so as to be able to participate in the interaction. For example, where the tumor homing peptide is suspected of being a ligand for a growth factor receptor, the binding portion of the molecule attached to a support is positioned so it can interact with the growth factor receptor on a cell in the tumor. If desired, an appropriate spacer molecule can be positioned between the molecule and the support such that the ability of the potential tumor homing molecule to interact with the target molecule is not hindered. A spacer molecule also can contain a reactive group, which provides a convenient and efficient means of linking a molecule to a support and, if desired, can contain a tag, which can facilitate recovery or identification of the molecule (see Hermanson, supra, 1996).

A peptide suspected of being able to home to a selected tumor such as Kaposi's Sarcoma, breast carcinoma or a melanoma can expressed as the N-terminus of a fusion protein, wherein the C-terminus consisted of a phage coat protein. Upon expression of the fusion protein, the C-terminal coat protein linked the fusion protein to the surface of a phage such that the N-terminal peptide was in a position to interact with a target molecule in the tumor. Thus, a molecule having a shared tag was formed by the linking of a peptide to a phage, wherein the phage provided a biological support, the peptide molecule was linked as a fusion protein, the phage-encoded portion of the fusion protein acted as a spacer molecule, and the nucleic acid encoding the peptide provided a specific tag allowing identification of a tumor homing peptide.

In vivo panning, which can be used to identify a tumor homing peptide, is means a method of screening a library by administering the library to a subject and identifying a molecule that selectively homes to a tumor in the subject (see U.S. Pat. No. 5,622,699). The terms “administering to an individual” or “administering to a subject,” when used in reference to a homing conjugate is used in its broadest sense to mean that the library is delivered to a tumor in the subject, which, generally, is a vertebrate, particularly a mammal such as a human.

A therapeutically effective amount of a homing conjugate or a library of candidate tumor homing peptide monomers or candidate tumor homing molecule dimers can be administered to a subject, for example, by injection into the circulation of the subject such that the molecules pass through the tumor. If desired, after an appropriate period of time, circulation can be terminated by sacrificing the subject or by removing a sample of the tumor (see, also, U.S. Pat. No. 5,622,699; Pasqualini and Ruoslahti, supra, 1996). Alternatively, a cannula can be inserted into a blood vessel in the subject, such that the molecules are administered by perfusion for an appropriate period of time. Similarly, a library can be shunted through one or a few organs, including the tumor, by cannulation of the appropriate blood vessels in the subject. It is recognized that a homing conjugate or a library of candidate tumor homing peptide monomers or candidate tumor homing molecule dimers also can be administered to an isolated perfused tumor. In particulare, panning in an isolated perfused tumor can be useful to identify molecules that bind to the tumor and, if desired, can be used as an initial screening of a library.

As described, in vivo panning can be used to identify tumor a homing peptide by screening a phage peptide display library in tumor-bearing model organisms and identifying specific peptides that selectively home to a tumor, for example, a breast tumor or to a melanoma. However, phage libraries that display protein receptor molecules, including, for example, an antibody or an antigen binding fragment of an antibody such an Fv, Fd or Fab fragment; a hormone receptor such as a growth factor receptor; or a cell adhesion receptor such as an integrin or a selectin also can be used to identify homing peptides. Variants of such molecules can be constructed using well known methods such as random mutagenesis, site-directed mutagenesis or codon based mutagenesis (see Huse, U.S. Pat. No. 5,264,563, issued Nov. 23, 1993, which is incorporated herein by reference). If desired, peptides can be dimerized following expression of the phage but prior to administration to the subject. Thus, various types of phage display libraries can be screened by in vivo panning.

Phage display technology provides a means for expressing a diverse population of random or selectively randomized peptides. Various methods of phage display and methods for producing diverse populations of peptides are well known in the art. For example, Ladner et al. (U.S. Pat. No. 5,223,409, issued Jun. 29, 1993, which is incorporated herein by reference) describe methods for preparing diverse populations of binding domains on the surface of a phage. In particular, Ladner et al. describe phage vectors useful for producing a phage display library, as well as methods for selecting potential binding domains and producing randomly or selectively mutated binding domains.

Similarly, Smith and Scott (Meth. Enzymol. 217:228-257 (1993); see, also, Scott and Smith, Science 249: 386-390 (1990), each of which is incorporated herein by reference) describe methods of producing phage peptide display libraries, including vectors and methods of diversifying the population of peptides that are expressed (see, also, Huse, WO 91/07141 and WO 91/07149, each of which is incorporated herein by reference). Phage display technology can be particularly powerful when used, for example, with a codon based mutagenesis method, which can be used to produce random peptides or randomly or desirably biased peptides (Huse, U.S. Pat. No. 5,264,563, supra, 1993). These or other well known methods can be used to produce a phage display library, which can be subjected to in vivo panning in order to identify tumor homing molecules useful in the homing pro-apoptotic conjugates of the invention.

In vivo panning provides a method for directly identifying tumor homing molecules that can selectively home to a tumor. As used herein, the term “home” or “selectively home” means that a particular molecule binds relatively specifically to a target molecule present in the tumor following administration to a subject. In general, a tumor homing molecule is characterized, in part, by exhibiting at least a two-fold (2×) greater specific binding to a tumor as compared to a control organ or tissue. Selective homing of a tumor homing molecule can be distinguished from nonspecific binding, however, by detecting differences in the abilities of different individual phage to home to a tumor. For example, selective homing can be identified by combining a putative tumor homing molecule such as a peptide expressed on a phage with a large excess of non-infective phage or with about a five-fold excess of phage expressing unselected peptides, injecting the mixture into a subject and collecting a sample of the tumor. In the latter case, for example, provided the number of injected phage expressing tumor homing peptide is sufficiently low so as to be nonsaturating for the target molecule, a determination that greater than about 20% of the phage in the tumor express the putative tumor homing molecule is demonstrative evidence that the peptide expressed by the phage is a specific tumor homing molecule. In addition, nonspecific localization can be distinguished from selective homing by performing competition experiments using, for example, phage expressing a putative tumor homing peptide in combination with an excess amount of the “free” peptide.

Selective homing of a tumor homing molecule can be demonstrated by determining the specificity of a tumor homing molecule for the tumor as compared to a control organ or tissue. Selective homing also can be demonstrated by showing that molecules that home to a tumor, as identified by one round of in vivo panning, are enriched for tumor homing molecules in a subsequent round of in vivo panning.

Tumor homing molecules can be identified by in vivo panning using, for example, a mouse containing a transplanted tumor. Such a transplanted tumor can be, for example, a human tumor that is transplanted into immunodeficient mice such as nude mice or a murine tumor that is maintained by passage in tissue culture or in mice. Due to the conserved nature of cellular receptors and of ligands that bind a particular receptor, it is expected that angiogenic vasculature and histologically similar tumor cells in various species can share common cell surface markers useful as target molecules for a tumor homing molecule. Thus, the skilled artisan would recognize that a tumor homing molecule identified using, for example, in vivo panning in a mouse having a murine tumor of a defined histological type such as a melanoma also would bind to the corresponding target molecule in a tumor in a human or other species. Similarly, tumors growing in experimental animals require associated neovascularization, just as that required for a tumor growing in a human or other species. Thus, a tumor homing molecule that binds a target molecule present in the vasculature in a tumor grown in a mouse likely also can bind to the corresponding target molecule in the vasculature of a tumor in a human or other mammalian subject. The general ability of a tumor homing molecule identified, for example, by homing to a human breast tumor, also to home to a human Kaposi's sarcoma or to a mouse melanoma indicates that the target molecules are shared by many tumors.

A tumor homing molecule identified using in vivo panning in an experimental animal such as a mouse readily can be examined for the ability to bind to a corresponding tumor in a human patient by demonstrating, for example, that the molecule also can bind specifically to a sample of the tumor obtained from the patient. For example, NGR peptides have been shown to bind to blood vessels in microscopic sections of human tumors, whereas little or no binding occurs in the blood vessels of nontumor tissues. Thus, routine methods can be used to confirm that a tumor homing molecule identified using in vivo panning in an experimental animal also can bind the target molecule in a human tumor.

Additional rounds of in vivo panning can be used to determine whether a particular molecule homes only to the selected tumor or can recognize a target on the tumor that also is expressed in one or more normal organs or tissues in a subject or is sufficiently similar to the target molecule on the tumor. It is unlikely that a tumor homing molecule also will home to a corresponding normal tissue because the method of in vivo panning selects only those molecules that home to the selected tumor. Where a tumor homing molecule also directs homing to one or more normal organs or tissues in addition to the tumor, the organs or tissues are considered to constitute a family of selected organs or tissues. Using the method of in vivo panning, molecules that home to only the selected tumor can be distinguished from molecules that also home to one or more selected organs or tissues. Such identification is expedited by collecting various organs or tissues during subsequent rounds of in vivo panning.

In vitro screening of phage libraries previously has been used to identify peptides that bind to antibodies or to cell surface receptors (Smith and Scott, supra, 1993). For example, in vitro screening of phage peptide display libraries has been used to identify novel peptides that specifically bound to integrin adhesion receptors (Koivunen et al., J. Cell Biol. 124:373-380 (1994a), which is incorporated herein by reference) and to the human urokinase receptor (Goodson et al., Proc. Natl. Acad. Sci., USA 91:7129-7133 (1994)). However, such in vitro studies provide no insight as to whether a peptide that can specifically bind to a selected receptor in vitro also will bind the receptor in vivo or whether the binding peptide or the receptor are unique to a specific organ in the body. Furthermore, the in vitro methods are performed using defined, well-characterized target molecules in an artificial system. For example, Goodson et al., supra, 1994, utilized cells expressing a recombinant urokinase receptor. However, such in vitro methods are limited in that they require prior knowledge of the target molecule and yield little if any information regarding in vivo utility.

In vitro panning against cells in culture also has been used to identify molecules that can specifically bind to a receptor expressed by the cells (Barry et al., Nature Med. 2:299-305 (1996), which is incorporated herein by reference). However, the cell surface molecules that are expressed by a cell in vivo often change when the cell is grown in culture. Thus, in vitro palming methods using cells in culture also are limited in that there is no guarantee a molecule that is identified due to its binding to a cell in culture will have the same binding ability in vivo. Furthermore, it is not possible to use in vitro panning to distinguish molecules that home only to the tumor cells used in the screening, but not to other cell types.

In contrast, in vivo panning requires no prior knowledge or availability of a target molecule and identifies molecules that bind to cell surface target molecules that are expressed in vivo. Also, since the “nontargeted” tissues are present during the screening, the probability of isolating tumor homing molecules that lack specificity of homing is greatly reduced. Furthermore, in obtaining tumor homing molecules by in vivo panning, any molecules that may be particularly susceptible to degradation in the circulation in vivo due, for example, to a metabolic activity, are not recovered. Thus, in vivo panning provides significant advantages over previous methods by identifying tumor homing molecules that selectively home in vivo to a target molecule present in a tumor. Evidence indicates, for example, that the vascular tissues in various organs differ from one another and that such differences can be involved in regulating cellular trafficking in the body. For example, lymphocytes home to lymph nodes or other lymphoid tissues due, in part, to the expression of specific “address” molecules by the endothelial cells in those tissues (Salmi et al., Proc. Natl. Acad. Sci., USA 89:11436-11440 (1992); Springer, Cell 76:301-314 (1994)). Similarly, various leukocytes can recognize sites of inflammation due, in part, to the expression of endothelial cell markers induced by inflammatory signals (see Butcher and Picker, Science 272:60-66 (1996); Springer, supra, 1994). Thus, endothelial cell markers provide a potential target that can be selectively bound by a tumor homing molecule and used to direct a linked antimicrobial peptide to a tumor.

Additional components can be included as part of the homing pro-apoptotic conjugate, if desired. For example, in some cases, it can be desirable to utilize an oligopeptide spacer between a tumor homing molecule and the antimicrobial peptide. Such spacers are well known in the art, as described, for example, in Fitzpatrick and Garnett, Anticancer Drug Des. 10:1-9 (1995)).

A homing conjugate of the invention can readily be synthesized in required quantities using routine methods of solid state peptide synthesis. A homing conjugate of the invention also can be purchased from a commercial source (for example, AnaSpec, Inc.; San Jose, Calif.). Several methods to link an antimicrobial peptide to a tumor homing peptide monomer are known in the art, depending on the particular chemical characteristics of the molecule. For example, methods of linking haptens to carrier proteins as used routinely in the field of applied immunology (see, for example, Harlow and Lane, supra, 1988; Hermanson, supra, 1996).

A premade antimicrobial peptide also can be conjugated to a tumor homing peptide monomer using, for example, carbodiimide conjugation (Bauminger and Wilchek, Meth. Enzymol. 70:151-159 (1980), which is incorporated herein by reference). Carbodiimides comprise a group of compounds that have the general formula R—N═C═N—Rα, where R and Rα can be aliphatic or aromatic, and are used for synthesis of peptide bonds. The preparative procedure is simple, relatively fast, and is carried out under mild conditions. Carbodiimide compounds attack carboxylic groups to change them into reactive sites for free amino groups. Carbodiimide conjugation has been used to conjugate a variety of compounds to carriers for the production of antibodies. The water soluble carbodiimide, 1-ethyl-3-(3-dimethylaminopropyl) carbodiimide (EDC) can be useful for conjugating an antimicrobial peptide to a tumor homing peptide monomer. Such conjugation requires the presence of an amino group, which can be provided, for example, by an antimicrobial peptide, and a carboxyl group, which can be provided by the tumor homing molecule.

In addition to using carbodiimides for the direct formation of peptide bonds, EDC also can be used to prepare active esters such as N-hydroxysuccinimide (NHS) ester. The NHS ester, which binds only to amino groups, then can be used to induce the formation of an amide bond with the single amino group of the doxorubicin. The use of EDC and NHS in combination is commonly used for conjugation in order to increase yield of conjugate formation (Bauminger and Wilchek, supra, 1980).

The yield of antimicrobial peptide/tumor homing molecule conjugate formed is determined using routine methods. For example, HPLC or capillary electrophoresis or other qualitative or quantitative method can be used (see, for example, Liu et al., J. Chromatogr. 735:357-366 (1996); Rose et al., J. Chromatogr. 425:419-412 (1988), each of which is incorporated herein by reference). In particular, the skilled artisan will recognize that the choice of a method for determining yield of a conjugation reaction depends, in part, on the physical and chemical characteristics of the specific antimicrobial peptide and tumor homing molecule. Following conjugation, the reaction products are desalted to remove any free peptide or molecule.

The present invention also provides methods of directing an antimicrobial peptide in vivo to a tumor having angiogenic vasculature. The method is practiced by administering a homing conjugate of the invention, for example, HK-1, to a subject containing a tumor having angiogenic vasculature. In a method of the invention for directing an antimicrobial peptide in vivo to a tumor having angiogenic vasculature, the antimicrobial peptide can include, for example, the sequence _{d}(KLAKLAK)₂ (SEQ ID NO: 15), or_{d}(ALLLAIRRR) (SEQ ID NO: 18) or _{d}(ALLLAIRRRKKK) (SEQ ID NO: 19). Particularly useful conjugates that can be administered to a subject containing a tumor having angiogenic vasculature include, for example, (CNGRC-GG-_{d}(KLAKLAK)₂)₂ (SEQ ID NO: 9), (CNGRC-GG-_{d}(ALLLAIRRR))₂ (SEQ ID NO: 10), and (CNGRC-GG-_{d}(ALLLAIRRRKKK))₂. (SEQ ID NO: 11).

The present invention additionally provides methods of inducing selective toxicity in vivo in a tumor having angiogenic vasculature. The methods are practiced by administering a homing conjugate of the invention, for example, HK-1, to a subject containing a tumor having angiogenic vasculature. An antimicrobial peptide useful in inducing selective toxicity in a method of the invention can be, for example, a peptide containing the sequence _{d}(KLAKLAK)₂ (SEQ ID NO: 15). Particularly useful conjugates that can be administered to induce selective toxicity in vivo in a tumor having angiogenic vasculature include (CNGRC-GG-_{d}(KLAKLAK)₂)₂ (SEQ ID NO: 9).

Also provided herein are methods of treating a patient with a tumor having angiogenic vasculature. In such methods of treatment, a homing conjugate of the invention is administered to the patient and is selectively toxic to the tumor. The antimicrobial peptide portion can include, for example, the sequence _{d}(KLAKLAK)₂ (SEQ ID NO: 15), or _{d}(ALLLAIRRR) (SEQ ID NO: 18) or _{d}(ALLLAIRRRKKK) (SEQ ID NO: 19). In preferred embodiments, the homing pro-apoptotic conjugate has the sequence (CNGRC-GG-_{d}(KLAKLAK)₂)₂ (SEQ ID NO: 9). In additional embodiments, the homing pro-apoptotic conjugate has the sequence (CNGRC-GG-_{d}(ALLLAIRRR))₂ (SEQ ID NO: 10). In further embodiments, the homing pro-apoptotic conjugate has the sequence (CNGRC-GG-_{d}(ALLLAIRRRKKK))₂ (SEQ ID NO: 11).

When administered to a subject, a homing conjugate of the invention can be administered as a pharmaceutical composition containing, for example, the conjugate and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters.

A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans; antioxidants, such as ascorbic acid or glutathione; chelating agents; low molecular weight proteins; or other stabilizers or excipients. One skilled in the art would know that the choice of a pharmaceutically acceptable carrier, including a physiologically acceptable compound, depends, for example, on the route of administration of the composition. The pharmaceutical composition also can contain an agent such as a cancer therapeutic agent.

One skilled in the art would know that a homing conjugate of the invention can be administered as a pharmaceutical composition to a subject by various routes including, for example, orally or parenterally, such as intravenously. A pharmaceutical composition containing the conjugate can be administered by injection or by intubation. The pharmaceutical composition also can be a tumor homing molecule linked to liposomes or other polymer matrices, which can have incorporated therein, an antimicrobial peptide (Gregoriadis, Liposome Technology, Vol. 1 (CRC Press, Boca Raton, Fla. 1984), which is incorporated herein by reference). Liposomes, for example, which consist of phospholipids or other lipids, are nontoxic, physiologically acceptable and metabolizable carriers that are relatively simple to make and administer.

For the therapeutic methods disclosed herein, an effective amount of the homing conjugate must be administered to the subject. As used herein, the term “effective amount” means the amount of the conjugate that produces the desired effect. An effective amount often will depend on the particular antimicrobial peptide linked to the tumor homing molecule. An effective amount of a homing pro-apoptotic conjugate in which a tumor homing molecule is linked to a particular antimicrobial peptide can be determined using methods well known to those in the art.

The route of administration of a homing conjugate depends, in part, on the chemical structure of the molecule. Peptides, for example, are not particularly useful when administered orally because they can be degraded in the digestive tract. However, methods for chemically modifying peptides to render them less susceptible to degradation by endogenous proteases or more absorbable through the alimentary tract, including incorporation of D-amino acids, are well known (see, for example, Blondelle et al., supra, 1995; Ecker and Crooke, supra, 1995; Goodman and Ro, supra, 1995). Such modifications can be performed on tumor homing peptides identified by in vivo panning as well as on antimicrobial peptides. In addition, methods for preparing libraries of peptidomimetics, which can contain D-amino acids, other non-naturally occurring amino acids, or chemically modified amino acids; or can be organic molecules that mimic the structure of a peptide; or can be peptoids such as vinylogous peptoids, are known in the art and can be used to identify tumor homing molecules that are stable for oral administration.

A tumor homing molecule tumor homing molecule is a dimer consisting of two tumor homing peptide monomers. Cysteine residues were included in some peptides, allowing dimerization of the peptide monomers. In particular, peptide monomers containing at cysteine residues dimerize spontaneously. In addition, such cyclic peptides also can be active when present in a linear form (see, for example, Koivunen et al., supra, 1993). Thus, in some cases one or more cysteine residues in the tumor homing peptide monomers can be deleted without significantly affecting the tumor homing activity of the homing conjugate provided the monomers can still dimerize to form the tumor homing molecule. Methods for determining the necessity of a cysteine residue or of amino acid residues N-terminal or C-terminal to a cysteine residue for tumor homing activity of a homing conjugate of the invention are routine and well known in the art.

Some, but not all, tumor homing molecules also can home to angiogenic vasculature that is not contained within a tumor. For example, tumor homing molecules containing either the RGD motif or the GSL motif specifically homed to retinal neovasculature (Smith et al., Invest. Ophthamol. Vis. Sci. 35:101-111 (1994), which is incorporated herein by reference), whereas tumor homing peptides containing the NGR motif did not accumulate substantially in this angiogenic vasculature. Therefore, tumor vasculature appears to express target molecules that are not substantially expressed by other kinds of angiogenic vasculature. Methods as disclosed herein can be used to distinguish tumor homing peptides from peptides that home to nontumor angiogenic vasculature. One skilled in the art understands that, preferably, for treatment of a tumor, one administers a conjugate having a tumor homing peptide, which selectively homes to tumor vasculature.

The invention provides a homing conjugate that includes a dimer of two endothelium-homing peptide monomers, at least one of which is linked to an antimicrobial peptide sequence has greatly increased pro-apoptotic activity compared to a monomeric homing conjugate. A homing conjugate of the invention generally is non-toxic outside of eukaryotic cells, but promotes disruption of mitochondrial membranes and subsequent cell death when targeted and internalized by eukaryotic cells. Homing conjugates such as (CNGRC-GG-_{d}(KLAKLAK)₂)₂, (SEQ ID NO.: 9) which contains the two copies of the antimicrobial peptide-_{d}(KLAKLAK)₂, (SEQ ID NO: 15) each linked to one monomer of the dimeric endothelium homing molecule (CNGRC)₂ (SEQ ID NO: 2), can have selective toxicity against angiogenic endothelial cells in vivo and, thus, can be used to treat, for example, benign hyperplasias or cancer. As disclosed herein, a dimer consisting of monomers containing the CNGRC peptide (SEQ ID NO: 1) can selectively localize to angiogenic endothelial tissue, specifically tumor vasculature, when systemically administered. Furthermore, a tumor homong molecule dimer consisting of monomers of the endothelium-homing peptide CNGRC (SEQ ID NO: 1) can be used to selectively deliver a linked moiety, such as biotin or phage, to angiogenic endothelial tissue.

Thus, the present invention provides a homing conjugate that includes a dimer of two endothelium-homing peptide monomers, at least one of which is linked to an antimicrobial peptide sequence, where the homing conjugate is selectively internalized by angiogenic endothelial tissue and exhibits high toxicity thereto, while the antimicrobial peptide has low mammalian cell toxicity when not linked to the endothelium-homing peptide. In a homing conjugate of the invention, the endothelium-homing peptide portion can contain, for example, the sequence CNGRC (SEQ ID NO: 1) or a functionally equivalent sequence, and the antimicrobial peptide portion can have an amphipathic α-helical structure such as the sequence _{d}(KLAKLAK)₂ (SEQ ID NO: 15), _{d}(ALLLAIRRR) (SEQ ID NO: 18) or _{d}(ALLLAIRRRKKK) (SEQ ID NO: 19).

In a preferred embodiment, the antimicrobial peptide portion contains the sequence _{d}(KLAKLAK)₂ (SEQ ID NO: 15). An exemplary endothelium-homing pro-apoptotic peptide is provided herein as (CNGRC-GG-_{d}(KLAKLAK)₂)₂ (SEQ ID NO: 9). In an additional embodiment, the antimicrobial peptide portion contains the sequence _{d}(ALLLAIRRR) (SEQ ID NO: 18). An exemplary endothelium-homing pro-apoptotic peptide is provided herein as (CNGRC-GG-_{d}(ALLLAIRRR))₂ (SEQ ID NO: 13). In a further embodiment, the antimicrobial peptide portion contains the sequence _{d}(ALLLAIRRRKKK) (SEQ ID NO: 19). An exemplary endothelium-homing pro-apoptotic peptide is provided herein as (CNGRC-GG-_{d}(ALLLAIRRRKKK))₂ (SEQ ID NO: 11).

The present invention further provides a method of directing an antimicrobial peptide in vivo to an angiogenic endothelial cell type or tissue. The method includes the step of administering a homing conjugate that includes a dimer of two endothelium-homing peptide monomers, at least one of which is linked to an antimicrobial peptide sequence, where the homing conjugate is selectively internalized by angiogenic endothelial tissue and exhibits high toxicity thereto, while the antimicrobial peptide has low mammalian cell toxicity when not linked to the endothelium-homing peptide. In a method of the invention, the endothelium-homing peptide can contain, for example, the sequence CNGRC (SEQ ID NO: 1) or a functionally equivalent sequence, and the antimicrobial peptide can contain a sequence such as _{d}(KLAKLAK)₂ (SEQ ID NO: 15) or _{d}(ALLLAIRRRR) (SEQ ID NO: 18) or _{d}(ALLLAIRRRRKKK) (SEQ ID NO: 19). In a preferred embodiment, the chimeric endothelium-homing pro-apoptotic peptide includes the sequence (CNGRC-GG-_{d}(KLALAK)₂)₂ (SEQ ID NO:12).

Also provided by the invention is a method of inducing selective toxicity in vivo in an angiogenic endothelial cell type or tissue. The method includes the step of administering to a subject containing a cancer a homing conjugate that includes a dimer of two endothelium-homing peptide monomers, at least one of which is linked to an antimicrobial peptide sequence, where the homing conjugate is selectively internalized by an angiogenic endothelial cell type or tissue and exhibits high toxicity thereto, while the antimicrobial peptide has low mammalian cell toxicity when not linked to the endothelium-homing peptide. The method of inducing selective toxicity in vivo in an angiogenic endothelial cell type or tissue can be practiced, for example, with a endothelium-homing peptide containing the sequence CNGRC (SEQ ID NO: 1) or a functionally equivalent sequence, and the antimicrobial peptide can contain a sequence such as _{d}(KLAKLAK)₂ (SEQ ID NO: 15), _{d}(ALLLAIRRRR) (SEQ ID NO: 18) or _{d}(ALLLAIRRRRKKK) (SEQ ID NO: 19). In a preferred embodiment, the chimeric endothelium-homing pro-apoptotic peptide includes the sequence (CNGRC-GG-_{d}(KLAKLAK)₂)₂ (SEQ ID NO: 9). In further embodiments, the chimeric endothelium-homing pro-apoptotic peptide includes the sequence (CNGRC-GG-_{d}(ALLLAIRRR))₂ (SEQ ID NO: 10) and (CNGRC-GG-_{d}(ALLLAIRRRKKK))₂ (SEQ ID NO: 11). In additional embodiments, the chimeric endothelium-homing pro-apoptotic peptide includes the sequence (ACDCRGDCFCG-GG-_{d}(KLAKLAK)₂)₂ (SEQ ID NO: 12), (ACDCRGDCFCG-GG-_{d}(ALLLAIRRR))₂ (SEQ ID NO: 13) and (ACDCRGDCFCG-GG-_{d}(ALLLAIRRRKKK))₂ (SEQ ID NO: 14).

In addition, the invention provides a method of treating a patient having cancer by administering to the patient a chimeric endothelium-homing pro-apoptotic peptide of the invention, whereby the homing conjugate is selectively toxic to the tumor. The homing conjugate cancer a homing conjugate that includes a dimer of two endothelium-homing peptide monomers, at least one of which is linked to an antimicrobial peptide sequence, and the homing conjugate is selectively internalized by angiogenic endothelial tissue and exhibits high toxicity thereto, while the antimicrobial peptide has low mammalian cell toxicity when not linked to the endothelium-homing peptide. The endothelium-homing peptide portion can contain, for example, the sequence CNGRC (SEQ ID NO: 1), the sequence ACDCRGDCFCG (SEQ ID NO: 3) or a functionally equivalent sequence, and the antimicrobial peptide can contain a sequence such as _{d}(KLAKLAK)₂ (SEQ ID NO: 15) or _{d}(ALLLAIRRRR) (SEQ ID NO: 18). In a preferred embodiment, the chimeric endothelium-homing pro-apoptotic peptide includes the sequence (CNGRC-GG-_{d}(KLAKLAKK)₂)₂ (SEQ ID NO: 9).

As used herein, the term “endothelium-homing peptide” means a peptide that selectively homes in vivo to angiogenic endothelial tissue as compared to control tissue, such as brain. Such a peptide generally is characterized by at least a two-fold greater localization to prostatic tissue as compared to a control cell type or tissue. An endothelium-homing peptide can selectively home, for example, to tumor vasculature as compared to other cell types or other vasculature.

A homing conjugate that includes a dimer of two endothelium-homing peptide monomers, at least one of which is linked to an antimicrobial peptide sequence, is selectively delivered to the angiogenic endothelial tissue of a tumor due to the selective homing activity of the endothelium-homing peptide portion. A variety of endothelium-homing peptides are useful in the invention, including CNGRC (SEQ ID NO: 1) and ACDCRGDCFCG (SEQ ID NO: 3).

In one embodiment, the invention relies on a endothelium-homing molecule consisting of two homing peptide monomers which contain the sequence CNGRC (SEQ ID NO: 1), or a functionally equivalent sequence. The term “functionally equivalent sequence,” as used herein in reference to the sequence CNGRC (SEQ ID NO: 1), means a sequence that binds selectively to the endothelium blood vessels, and that functions similarly in that the sequence binds selectively to the same receptor.

In a separate embodiment, the invention relies on a endothelium-homing molecule consisting of two homing peptide monomers which contain the sequence ACDCRGDCFCG (SEQ ID NO: 3), or a functionally equivalent sequence. The term “functionally equivalent sequence,” as used herein in reference to the sequence ACDCRGDCFCG (SEQ ID NO: 3), means a sequence that binds selectively to the endothelium blood vessels, and that functions similarly in that the sequence binds selectively to the same receptor. The sequence ACDCRGDCFCG (SEQ ID NO: 3) is also referred to in the art and herein as “RGD4C.”

In embodiments of the invention that include the RGD4C homing peptide, the chimeric endothelium-homing pro-apoptotic peptide can include, for example, the sequences (ACDCRGDCFCG-GG-_{d}(KLALAK)₂)₂ (SEQ ID NO: 9), (ACDCRGDCFCG-GG-_{d}(ALLLAIRRR))₂ (SEQ ID NO: 13) and (ACDCRGDCFCG-GG-_{d}(ALLLAIRRR K))₂ (SEQ ID NO:14).

It is understood that the endothelium-homing molecules that include a dimer of two endothelium-homing peptide monomers can be used to induce selective toxicity in a variety of disorders. Such disorders include cancer as well as any other conditions associated with an increase in angiogenesis. As used herein, the term “angiogenic endothelial tissue” refers to proliferating blood vessels. Such angiogenic vessels are distinguishable from mature vasculature due, in part, to expression of unique endothelial cell surface markers, including the α₁β₃ integrin (Brooks, Cell 79:1157-1164 (1994); WO 95/14714, Int. Filing Date Nov. 22, 1994) and receptors for angiogenic growth factors (Mustonen and Alitalo, J. Cell Biol. 129:895-898 (1995); Lappi, Semin. Cancer Biol. 6:279-288 (1995)).

In one embodiment, a method of the invention is useful for treating cancer with a chimeric endothelium-homing pro-apoptotic peptide. The chimeric endothelium-homing pro-apoptotic peptide can be utilized to target tumor vasculature, which is the angiogenic vasculature that supports the growth or maintenance of a tumor, which may be malignant or non-neoplastic. Like other angiogenic vessels, tumor vasculature can express unique endothelial cell surface markers. Moreover, tumor vasculature is histologically distinguishable from other blood vessels in that tumor vasculature generally is fenestrated (Folkman, Nature Med. 1:27-31 (1995); Rak et al., Anticancer Drugs 6:3-18 (1995)).

A chimeric endothelium-homing pro-apoptotic peptide of the invention also can be directed to angiogenic endothelial tissue that is not tumor vasculature or associated with neoplastic disease. Angiogenesis within the female reproductive tract, for example, is critical for normal reproduction and can be involved in pathogenesis of endometriosis (Donnez et al., Human Reproduction 13:1686-1690 (1998). Thus, a method of the invention can be useful in directing a chimeric endothelium-homing pro-apoptotic peptide to non-tumor angiogenic vasculature such as endometrial vasculature. Neovascularization also has been described within the intima of human atherosclerotic lesions and, further, angiogenic inhibitors such as endostatin can reduce the intimal neovascularization and plaque growth evident in apolipoprotein E-deficient mice (Moulton et al., Circulation 99:1726-1732 (1999)). Thus, a method of the invention can be useful for directing a therapeutic moiety or imaging agent to angiogenic sites in atherosclerotic plaques. Unregulated angiogenesis also can be involved in other non-neoplastic diseases such as diabetic blindness and rheumatoid arthritis. Thus, a chimeric endothelium-homing pro-apoptotic peptide of the invention also can be useful for treating disorders involving tumor vasculature or other neovasculature such as the vasculature present in inflammatory or other disorders or the neovasculature present in regenerating or wounded tissue. The following example is intended to illustrate but not limit the present invention.

### EXAMPLE I

### Characterization of (CNGRC-GG-_{d}(KLAKLAK)₂

This example demonstrates that (CNGRC-GG-_{d}(KLAKLAK)₂)₂ (SEQ ID NO: 9) reduces mitochondrial function by 63 percent in Kaposi's sarcoma cells. The example also demonstrates that targeting of Kaposi's sarcoma cells with an antimicrobial peptide delivered by a tumor homing molecule that consists of a dimer of two endothelium-homing peptide monomers results in 50 percent cell death.

KS cells were plated in DMEM with 10% FBS in 96-well plates. Media was replaced with 100 uL of DMEM without serum and cells were treated with peptides at 12 ug/mL: The peptides utilized had two distinct disulfide links and are shown below. The other peptides utilized in the studies and methods for tumor studies are essentially as reported in Nature Medicine 5(9): 1032-1038, 1999, which is incorporated herein by reference in its entirety. Crude peptide was unpurified peptide composed monomer and dimer and other species-oxidized forms of the peptide.

In order to determine pro-apoptotic effectiveness of HK-1 in tumor cells, viability was measured using the Live/Dead assay (Molecular Probes) and the WST-1 assay (WST (tetrazolium salt (4-[3-(4-Iodophenyl)-2-(4-nitrophenyl)-2H-5-tetrazolio]-1,3-benzene disulfonate; Boehringer Mannheim, Cat. No. 1644807)), both according to manufacturer's instructions. The data depicted in FIG. 3 is representative of two-three experiments with all samples measured in triplicate. Statistical significance was determined using the one-way ANOVA. As shown in FIG. 3, Kaposi's sarcoma cells treated with HK-1, which has the structure (CNGRC-GG-_{d}(KLAKLAK)₂)₂, (SEQ ID NO: 9) show greatly increased pro-apoptotic activity, killing almost 50 percent of the treated cancer cells, compared to cell death of less than 10 percent observed upon treatment with the corresponding monomeric homing conjugate CNGRC-GG-_{d}(KLAKLAK)₂ (SEQ ID NO: 20), crude preparation of the CNGRC homing peptide monomers, and in untreated cells.

In order to determine the decrease in mitochondrial function in tumor cells targeted with HK-1, which has the structure (CNGRC-GG-_{d}(KLAKLAK)₂)₂ (SEQ ID NO: 9), Kaposi's sarcoma cells were treated with HK-1, with the corresponding monomeric homing conjugate CNGRC-GG-_{d}(KLAKLAK)₂ (SEQ ID NO: 20), with a crude preparation of the CNGRC homing peptide monomers, with an RGD peptide preparation, with a preparation including KLAKLAK (SEQ ID NO: 5) antimicrobial peptides, and with a CNGR control preparation. As shown in FIG. 2, the HK-1, which has the structure (CNGRC-GG-_{d}(KLAKLAK)₂)₂ (SEQ ID NO: 9), reduced mitochondrial function in KS cells by 63 percent compared to a 34 percent reduction with the corresponding monomeric homing conjugate CNGRC-GG-_{d}(KLAKLAK)₂ (SEQ ID NO: 20), and 36 percent reduction with the RGD peptide preparation.

This example demonstrates that HK-1 (CNGRC-GG-_{d}(KLAKLAK)₂)₂ (SEQ ID NO: 9), can reduces mitochondrial function and effects cell death in tumor cells with greatly increased efficiency compared to the corresponding monomeric homing conjugate.

We claim:

1. A homing conjugate, comprising an antimicrobial peptide and a tumor homing molecule,
wherein said tumor homing molecule comprises a dimer of two endothelium-homing peptide monomers,
wherein said conjugate homes to and is internalized by a tumor cell type or tissue comprising angiogenic endothelial cells and exhibits high toxicity thereto,
wherein said high toxicity is due to disruption of mitochondrial membranes,
and wherein said antimicrobial peptides have low mammalian cell toxicity when not linked to said tumor homing molecule.

2. The homing conjugate of claim 1, further comprising two antimicrobial peptides.

3. The homing conjugate of claim 1, wherein said endothelium-homing peptide comprises the sequence CNGRC (SEQ ID NO: 1).

4. The homing conjugate of claim 1, wherein said dimer comprises disulfide bonds between said endothelium-homing peptide monomers.

5. The homing conjugate of claim 1, wherein said antimicrobial peptide comprises the sequence _{d}(KLAKLAK)₂ (SEQ ID NO: 15).

6. The homing conjugate of claim 1, comprising the sequence (CNGRC-GG-_{d}(KLAKLAK)₂)₂ (SEQ ID NO: 9).

7. A method of directing a homing conjugate in vivo to an angiogenic endothelial tissue or cell type, comprising administering the homing conjugate of claim 1, 2 or 6.

8. The method of claim 7, wherein said angiogenic entothelial tissue or cell type is associated with cancer.

9. A method of inducing selective toxicity in vivo in an angiogenic endothelial tissue or cell type, comprising administering to an individual an effective amount of the homing conjugate of claim 1, 2 or 6.

10. The method of claim 9, wherein said angiogenic endothelial tissue or cell type is associated with cancer.

11. A method of treating an individual having cancer, comprising administering an effective amount of the homing conjugate of claim 1, 2 or 6 to said individual, whereby said homing conjugate is selectively toxic to a tumor.
